(19) United States
(12) Patent Application Publication
(10) Pub. No.: US 2006/0216724 A1
(43) Pub. Date: Sep. 28, 2006

(54) Methods for normalized amplification of nucleic acids

(75) Inventors: Frederick C. Christians, Los Altos Hills, CA (US); Sean Walsh, Danville, CA (US); Rui Mei, Santa Clara, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/192,854

(22) Filed: Jul. 29, 2005

Related U.S. Application Data
(60) Provisional application No. 60/592,511, filed on Jul. 30, 2004.

(51) Int. Cl. C12Q 1/68; C12P 19/34

(52) U.S. Cl. 435/6; 435/91.2

(57) ABSTRACT

Methods of preparing normalized mixtures from a plurality of nucleic acid samples are disclosed. Nucleic acids are amplified so that similar amounts of a target nucleic acid are generated in a plurality of different reactions. Separate amplification reactions are performed to amplify the same or different targets in a plurality of different reactions. The amounts of amplified product are approximately normalized during the amplification without the need to empirically measure the amount of amplified target.

### PRIORITY

The present application claims priority to U.S. Provisional Application No. 60/592,511 filed Jul. 30, 2004, the entire disclosure of which is incorporated herein by reference in its entirety for all purposes.

### FIELD OF THE INVENTION

The present invention relates to the field of nucleic acid analysis and methods for normalizing nucleic acid samples.

### BACKGROUND OF THE INVENTION

Many methods of nucleic acid analysis require that two or more different samples of nucleic acid be mixed into a single mixture prior to subsequent analysis. It is often useful and sometimes necessary to measure the amount of nucleic acid in each of the different samples before adding them to the mixture so that proportional quantities of nucleic acid are added to the mixture from each of the different samples. Taking empirical measurements to quantify the amount of nucleic acid in a given sample or to determine the amount of a specific nucleic acid in a sample can be time consuming and tedious. Also, once the amount of nucleic acid in a sample is quantified it is often necessary to add very different volumes of each sample to the mixture to obtain the desired ratio of nucleic acids in the mixture. For example, if a first sample is much more concentrated than a second sample it may be necessary to add a very small volume of the first sample and a relatively large volume of the second sample. This mixing of unequal volumes may result in errors in the final mixture because, for example, when transferring small volumes of liquid small errors in measurement can result in relatively large errors in the final mixture.

### SUMMARY OF THE INVENTION

In one embodiment a method of amplifying a target sequence from a complex nucleic acid sample is disclosed. The target is amplified from the complex nucleic acid sample in a first amplification reaction to generate a first amplification product that is enriched for said target. The first amplification reaction is a polymerase chain reaction and the target is amplified using a pair of primers that are specific for the target. The first amplification product is then amplified in a second amplification reaction using a strand displacing DNA polymerase, such as phi29 or Bst DNA polymerase. The yield of the reaction is limited by the amount of raw material in the reaction, for example, the amount of dNTPs and random primers. The result of the second amplification reaction is a second amplification product that is enriched for the target and has a predictable yield. The target is present in the second amplification reaction in amounts that are determined by the amount of dNTPs added to the reaction because essentially all of the dNTPs end up in amplified copies of the target. It is possible to predict the amount of target generated because the amount will be proportional to the amount of dNTPs. The number of moles of target in each I of the second amplification reaction may be estimated using the known or estimated molecular weight of the target.

In one embodiment a method of analyzing a nucleic acid sample is disclosed. A first target sequence is amplified by target specific PCR and the amplification product is amplified by strand displacement amplification with a polymerase such as phi29 or Bst DNA polymerase using random primers. The strand displacing enzyme is highly processive so the amplification reaction goes to completion, until the dNTPs run out. The amplified target from the PCR reaction is the predominant target present in the second reaction so the majority of the amplification product resulting from the second reaction is amplified target. In a preferred embodiment a plurality of targets are amplified in separate reactions. The amount of dNTPs present in the second amplification reactions of each target are approximately the same so the yields of the second reactions are similar and can be estimated without empirical measurement.

In another embodiment a plurality of targets are amplified according to the methods and aliquots of the second amplification reaction are pooled to form a pooled sample. For each target a volume from the second amplification reaction that is proportional to the molecular weight of the target is added to the pooled sample so after pooling the pooled sample has approximately equivalent molar amounts of each target. The pooled sample may be subjected to further analysis. In a preferred embodiment the pooled sample is fragmented, the fragments are labeled and hybridized to an array of probes. In a preferred aspect the array is a resequencing array for resequencing between 30 and 300 kb of sequence. The resequencing array may have a reference sequences and a plurality of possible single nucleotide variations, deletions or insertions in the reference sequence. The hybridization pattern may be analyzed to identify variations in the reference sequence in the sample from which the target was amplified.

In another embodiment a plurality of target sequences of lengths between 1 and 30 kilobases are pooled to form a pooled sample by mixing amounts of an amplification reaction that are proportional to the molecular weight of the target. The reactions are assumed to have the same yield of target. The yield may be, for example, 0.5 to 2 μg target DNA per μl of reaction volume. The volume to add to the pooled reaction is determined by the molecular weight of the target amplified in that reaction.

### BRIEF DESCRIPTION OF THE FIGURES

FIG. 1 shows a schematic of a method of normalization using a first target specific amplification and a second amplification with random hexamers.

FIG. 2 is a flow chart of a method of pooling approximately equal molar amounts of a plurality of targets without empirical measurement for analysis by hybridization.

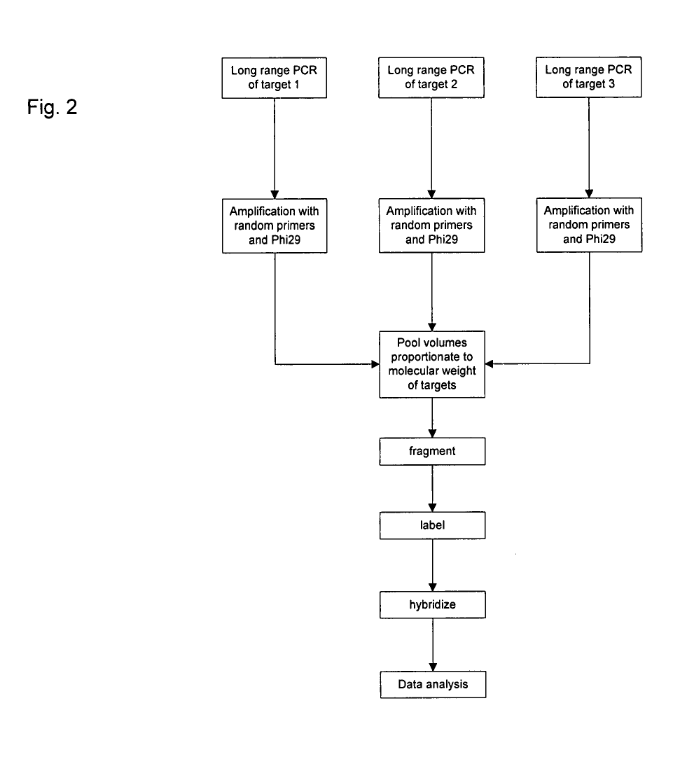

FIG. 3. Method for circularization of genomic fragments. FIG. 3A shows a single overhang adaptor which ligates to XbaI restriction fragments on either end. FIG. 3B shows an adaptor with XbaI overhangs on either end, which allows for circularization and concatenation of fragments.

FIG. 4 shows a schematic of the use of adaptors to circularize genomic fragments using a stem-loop adaptor. FIG. 4A shows the use of an adaptor that is a single molecule folded upon itself to form a sticky end and a step loop. The step-loop adaptor ligated to both ends of a fragment, followed by denaturation, generates a single stranded circular molecule.

FIG. 5 shows exponential amplification using a primer that is complementary to the adaptor.

FIG. 6 shows an example of how a restriction site may be engineered so that it is generated when two adaptors ligate together.

FIG. 7 shows a method for making single stranded circles from genomic fragments using partial adaptors.

FIG. 8 shows a method for amplifying single-stranded circles using rolling circle replication.

FIG. 9 shows a method of making single-stranded circles using a partial adaptor.

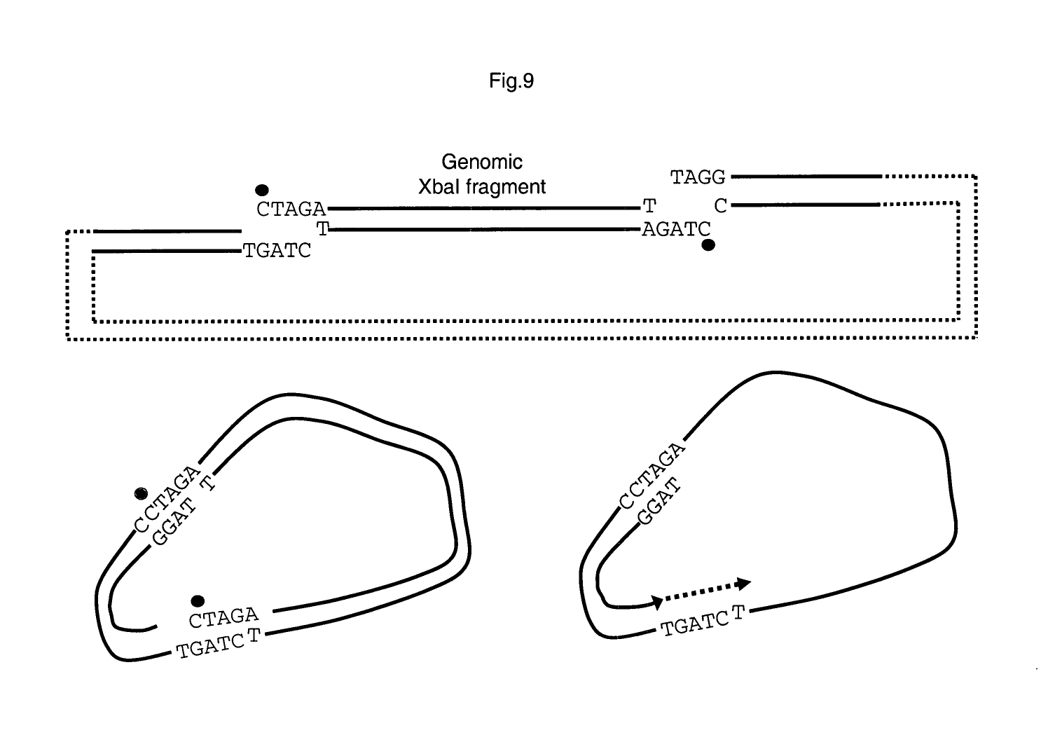

FIG. 10 shows a method of amplification of restriction fragments on a solid support using RCA.

FIG. 11 shows amplification of restriction fragments using branching rolling circle replication.

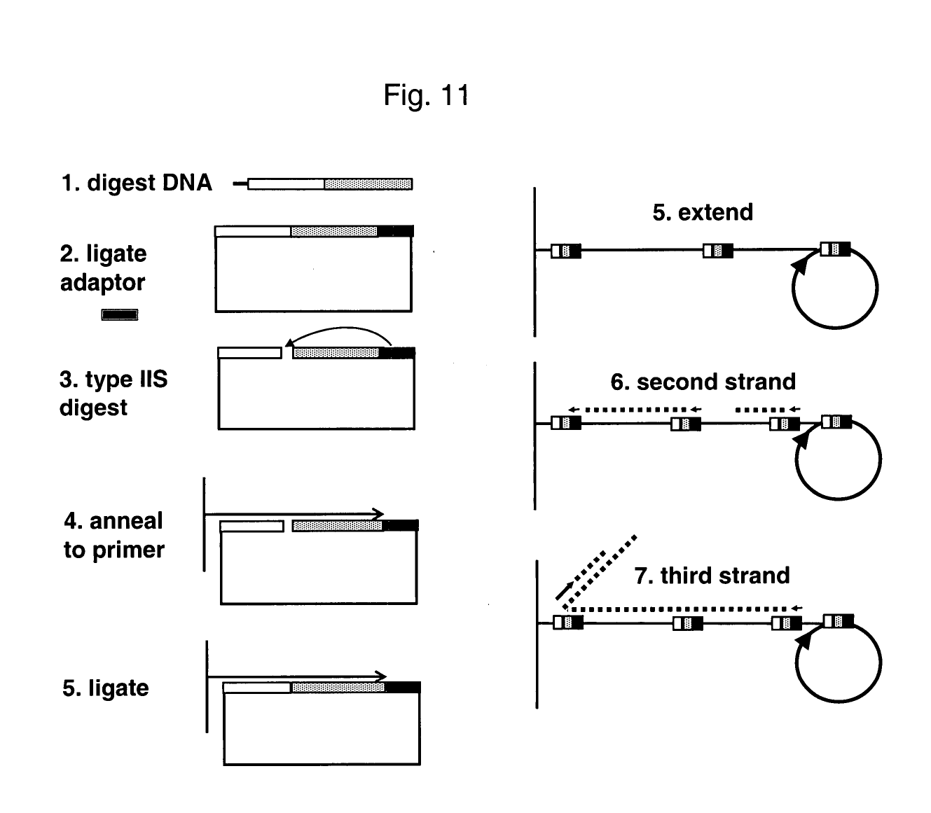

FIG. 12 shows amplification of restriction fragments using branching rolling circle replication using a 3′ to 5′ oligonucleotide array.

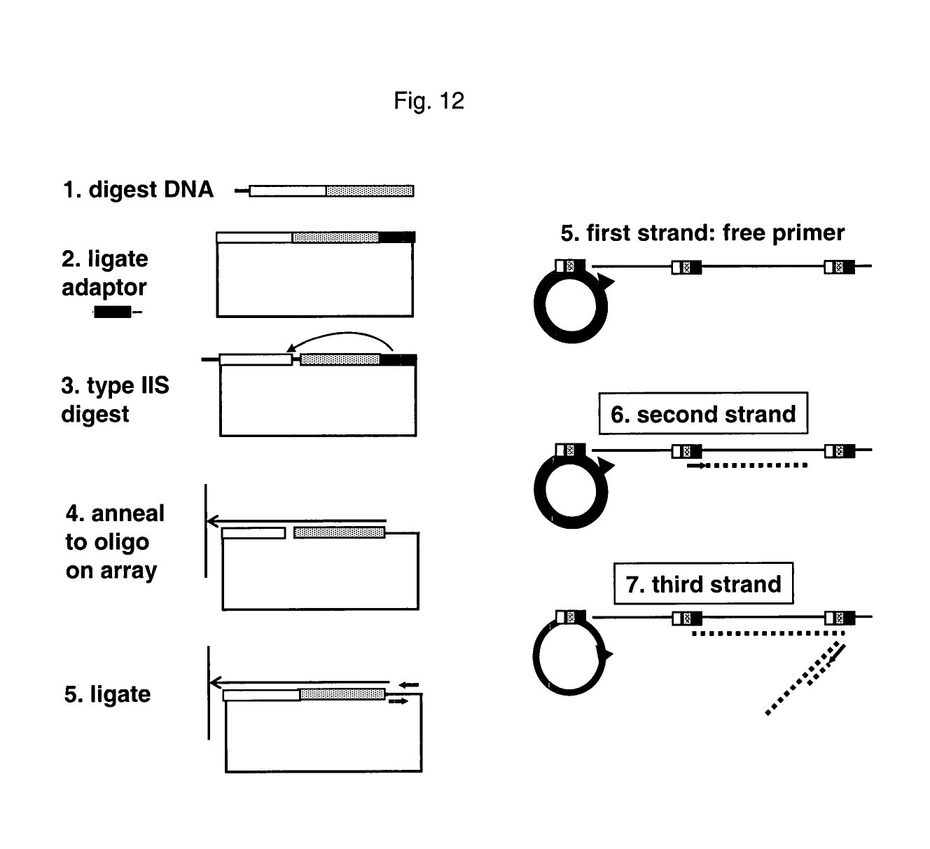

FIG. 13 shows circularization of restriction fragments on a 3′ to 5′ oligonucleotide array followed by PCR amplification.

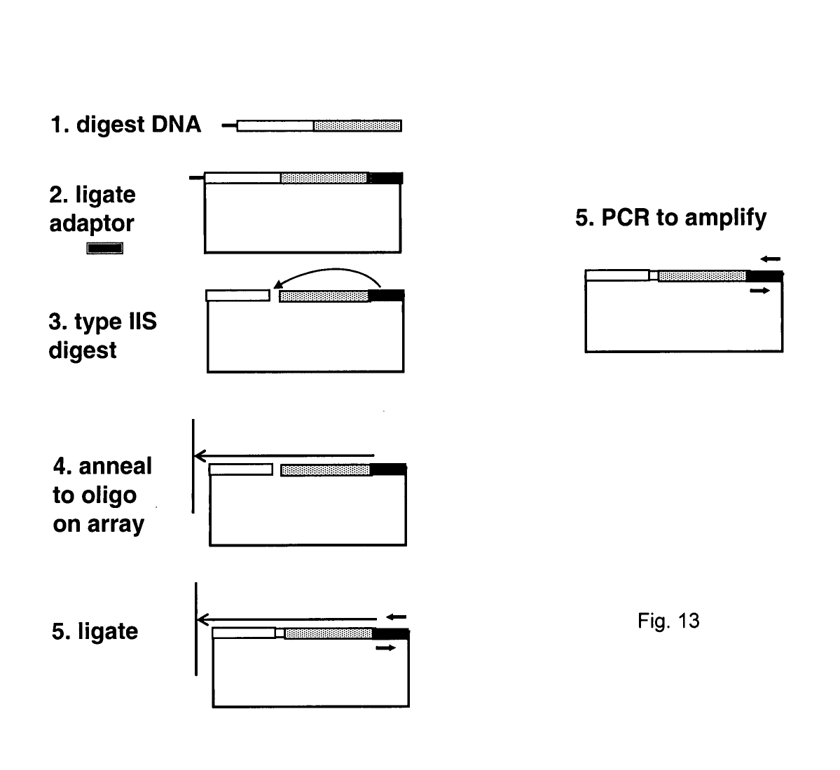

FIG. 14 shows SNP detection ligation discrimination with extension by RCA.

FIG. 15 shows SNP detection with an SBE reaction on an array.

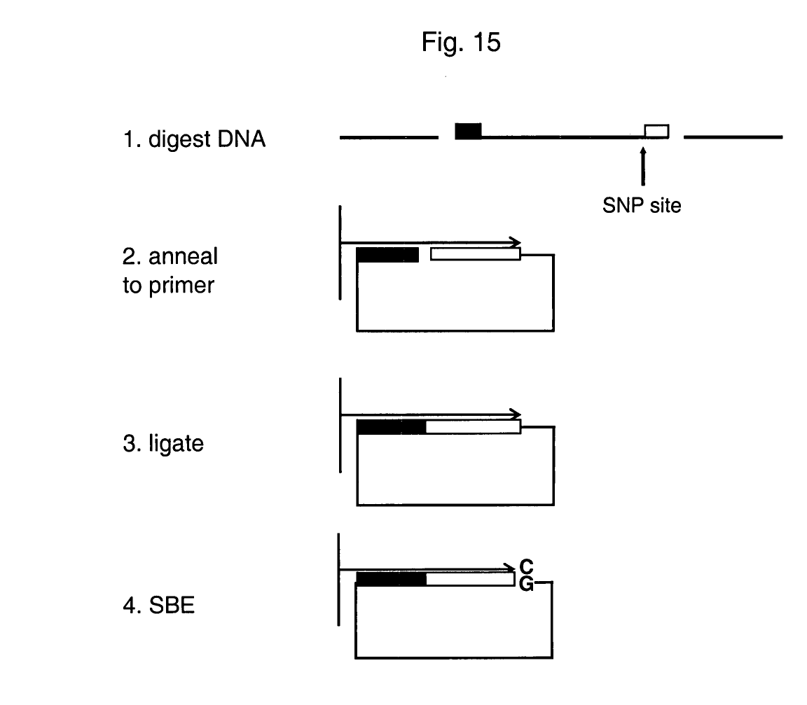

### DETAILED DESCRIPTION OF THE INVENTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909, 5,861,245) and nucleic acid based sequence amplification (NASBA). (See, U.S. Pat. Nos. 5,409,818, 5,554,517, and 6,063,603, each of which is incorporated herein by reference). Other amplification methods that may be used include: Qbeta Replicase, described in PCT Patent Application No. PCT/US87/00880, isothermal amplification methods such as SDA, described in Walker et al. 1992, Nucleic Acids Res. 20(7):1691-6, 1992, and rolling circle amplification, described in U.S. Pat. No. 5,648,245. Other amplification methods that may be used are described in, U.S. Pat. Nos. 5,242,794, 5,494,810, 4,988,617 and in U.S. Ser. No. 09/854,317, each of which is incorporated herein by reference. Other amplification methods that may be used are disclosed in US Patent Application Publication No. 20030143599.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication Number 20020183936), 10/065,856, 10/065,868, 10/328,818, 10/328,872, 10/423,403, and 60/482,389.

b) Definitions

The term “admixture” refers to the phenomenon of gene flow between populations resulting from migration. Admixture can create linkage disequilibrium (LD).

The term “allele” as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats,for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C, preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Methods for Automated Normalization of Target Samples

In general, methods for amplification and analysis of nucleic acid samples are disclosed. In preferred aspects the methods result in amplification of one or more targets so that a predictable concentration of amplified target results. The methods may be used to amplify a plurality of targets in a plurality of different reactions so that the amount of amplified target in each reaction is approximately the same. A plurality of the amplified targets can be mixed together to form a pooled sample with each target present at approximately equal concentrations.

When two or more target nucleic acids are to be pooled and analyzed as a pooled sample it is often desirable that an amount of each target is added to the pooled sample so that each target is present at approximately the same concentration in the pooled sample. This can be done by measuring the amount of nucleic acid in a sample after locus specific amplification (for example, by measuring the OD 260/280), calculating the molar concentration of each target based on the measurement and the calculated molecular weight of the target, determining the amount of each target that should be added to the pooled sample to provide the desired concentrations of each target (for example, approximately equal molar concentrations of several different targets) and aliquoting different volumes of each sample in order to provide a pooled sample where each target is present at the desired concentrations, (e.g. approximately equal molar concentrations), however, this method is tedious, time consuming and can introduce experimental error because, for example, it often requires transfer of small and unequal sample volumes.

The disclosed methods eliminate the need for measuring the concentration of amplified target in each reaction by employing a first PCR step that results in enrichment of a target in a sample and a second normalizing amplification that generates approximately the same amount (μg/μl) of amplification product in each reaction. The yield of the PCR reaction is variable, but is predominantly the target amplicon and the yield of the second reaction is approximately constant and is also predominantly the target amplicon. The second reaction generates approximately the same amount of product and by assuming that the amount in μg/μl is relatively constant the number of moles per μl can be estimated based on the predicted molecular weight of the target. In a preferred embodiment, the methods preferably eliminate the need to quantify the yield of nucleic acid in each individual sample empirically and the need to take highly variable volumes from each individual sample in order to add equivalent molar amounts of nucleic acid from each experimental sample to the pooled sample. The methods may be useful for target preparation for nucleic acid analysis methods including resequencing, genotype analysis, copy number analysis and gene expression analysis. In particularly preferred embodiments the targets prepared according to the disclosed methods are analyzed by hybridization to an array of nucleic acid probes. Methods for hybridization to arrays are well known in the art and are discussed in CustomSeq™]Resequencing Array Protocol, GeneChip® Expression Analysis Technical Manual and 100K Mapping Assay Manual, each of which is available from Affymetrix, Inc. Santa Clara and on the Affymetrix web site.

The methods are particularly useful for preparation of target for hybridization to resequencing arrays. Resequencing arrays may be designed to identify sequence variation in one or more genomic regions of interest. Depending on the feature size an array may be designed to detect variation from both strands of about 30 kb, about 300 kb or more. The sequence to be analyzed may be amplified by locus specific long range PCR in a plurality of individual reactions that each contain a single primer pair, although multiplex PCR may also be used. In a preferred embodiment the individual amplicons are about 5 to 10 kb, but may be between 1 and 30 kb or greater than 30 kb. Amplicons smaller than 1 kb may also be used. If the targets are all approximately the same length and molecular weight (plus or minu 10-20%), equal volumes of the second amplification reaction may be pooled to achieve equal molar amounts of the amplified targets. If two or more targets to be pooled vary in length by 2 fold or more the amount added for each target should be adjusted accordingly, for example, if one target is 5 kb and another is 1 kb in order to get the same molar amounts, assuming the total yield (in μg DNA per 1l) is the same, the volume used from the reaction for the larger target should be about 5× the volume used for the smaller target.

The efficiency of a PCR reaction can vary between samples. Assay performance on resequencing arrays may be compromised if amplicon concentration in the hybridization varies by more than two fold. Therefore to achieve the maximum amount of sequence information from a single hybridization, similar molar quantities of each target should be applied to the probe array. In a preferred embodiment each target amplicon is applied to the array at a concentration of about 200-500 picomolar and most preferably about 250 picomolar. Preferably the concentration of any two amplicons in the hybridization mixture varies by less than two fold.

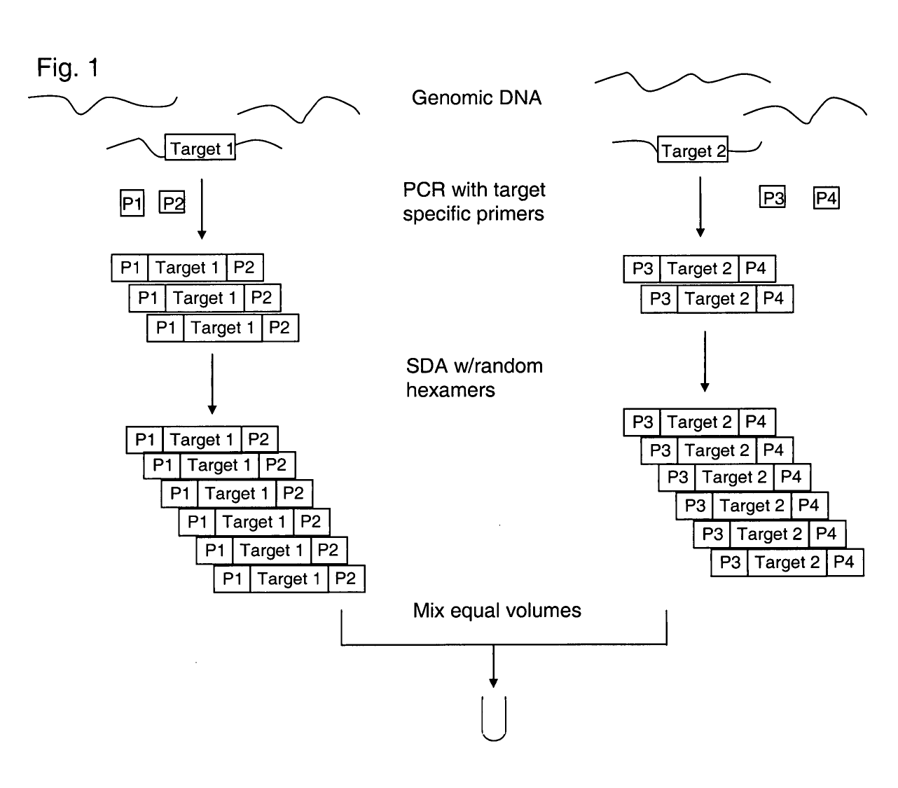

In one embodiment (FIG. 1), two samples are amplified in separate reactions. Target 1 is amplified in the first sample and target 2 in the second sample. In the first amplification target 1 is amplified by PCR using primers 1 and 2 which are specific for target 1 and in the second reaction target 2 is amplified by PCR using primers 3 and 4 which are specific for target 2. The primers may be locus specific primers, allele specific primers or primers that are complementary to adaptor sequences that are ligated to the ends of the target. After the targets are enriched relative to other sequences by the first target specific amplification, the sample is subjected to a second amplification step that using strand displacement amplification using non target specific primers. The primers may be random sequence primers, for example, random hexamers. Because the second amplification reaction continues until the nucleotides in the reaction are consumed, approximately the same amount of product will be generated. At the end of the second amplification reactions the amount of amplified target 1 is approximately the same as the amount of amplified target 2. Equal volumes of the reactions may be pooled in a new tube so that the new tube has approximately equal amounts of target 1 and target 2.

In a preferred embodiment each target to be pooled is amplified under conditions that are estimated to yield an approximately equal concentration of amplified target. Each individual amplification reaction may be, for example, limited to provide approximately the same yield by adding approximately the same concentration of dNTPs. In a preferred embodiment the yield of the amplification reaction is limited by the concentration of at least one dNTP added to the reaction so different concentrations of starting template may result in approximately the same concentration of amplified product in each of the individual amplification reactions. Equal amounts of a plurality of individual amplification reactions can be pooled to provide a pooled sample without empirically measuring the concentration of the amplified target in the individual amplification reactions.

In one embodiment individual nucleic acid samples containing one or more target nucleic acid are amplified prior to pooling under conditions where amplification yield is limited by the concentration of one or more of the components added to the amplification reaction. In a preferred embodiment the amplification yield is limited by the concentration of dNTPs in the amplification reaction and a highly processive polymerase is used for amplification. The yield of the amplified target or targets may be estimated based on the known concentration of dNTPs in the reaction.

In many embodiments, prior to the amplification step that is yield limiting the target may be amplified by a first amplification step that may be target specific. In particularly preferred embodiments targets are first amplified from a complex mixture, for example, genomic DNA, total RNA or polyA RNA, using an amplification method such as PCR or RT-PCR using one or more primers that are target specific. After this first amplification, the target is the most abundant amplified species in the reaction.

In one embodiment the methods of the present invention provide a simplified method for normalizing the amount of amplicon generated in primer mediated amplification reactions. This is particularly useful when a plurality of amplification reactions are performed in separate reactions and the amplification products are to be analyzed and compared or pooled and the pooled product analyzed.

It is often useful to pool products of two or more PCR reactions prior to a downstream analysis step. For example, if many targets are being amplified they can be amplified in two or more amplification reactions and the reactions may be pooled prior to analysis by methods such as hybridization to an array of nucleic acid probes. Because PCR amplification is exponential in nature the concentration of the amplified target in one reaction may differ significantly from the concentration of amplified target in a second reaction due to differences in the amount of target in each sample prior to amplification and to differences that may occur during amplification.

In one embodiment the concentration of target sequences is normalized by amplification with a phi-29 DNA polymerase. The yield of the amplification reaction is limited by the concentration of dNTP so the concentration of a single target in the amplified sample is the same regardless of the starting concentration. Whole genome amplification using multiple displacement amplification and related methods of assessment of MDA have also been disclosed in Dean et al. PNAS 99:5261-5266 (2002), Hosono et al. Genome Res. 13, 954-964 (2003) and Yan et al, Biotechniques 37, 136-143 (2004). A single stranded circular nucleic acid may be used as template.

In a preferred embodiment two or more long range locus specific PCR amplifications are performed, the reactions are subjected to amplification using phi-29 and random primers, an equivalent volume of each reaction is pooled into a single tube, the pooled sample is fragmented and labeled and hybridized to an array of nucleic acid probes that are complimentary to the amplified products and the hybridization pattern is analyzed to determine the presence or absence of target sequences. In a preferred embodiment the array of probes is a resequencing array with probes tiled to detect all possible single nucleotide variation in a reference sequence. For a description of resequencing arrays and methods of using resequencing arrays see, for example, U.S. Pat. Nos. 5,858,659, 5,925,525, 5,968,740, 6,268,141, 6,268,152 and 6,284,460, each of which is incorporated herein by reference in its entirety for all purposes.

In a preferred embodiment target sequences are amplified by PCR using sequence specific primers. The resulting amplified product which is enriched for the target sequences is then amplified using a strand displacing enzyme with high processivity, for example, Phi29. Phi29 is a highly processive DNA polymerase with high strand displacing activity. The enzyme is capable of extending long regions of DNA, for example, 10 kb fragments and greater. Variant forms of the enzyme are available, for example, exonuclease minus variants (see, for example, U.S. Pat. Nos. 5,001,050, 5,198,543, 5,854,033and 5,576,204). Phi 29 and methods of using phi29 have been described in numerous patents and publications. See, for example, U.S. Pat. Nos. 6,280,949 and 6,642,034 and Blanco, L. and Salas, M. (1984) Proc. Natl. Acad. Sci. USA, 81, 5325-5329, Blanco, et al. (1994) Proc. Natl. Acad. Sci. USA, 91, 12198-12202, Dean, et al. (2001) Genome Res., 11, 1095-1099, Blanco. L., et al., (1989) J. Biol. Chem., 264, 8935-8940, Garmendia, et al., (1992) J. Biol. Chem., 267, 2594-2599, and Lizardi, et al., (1998) Nature Genet., 19, 225-232. Additional information about phi 29 may be found in the following publications: Gen. Res., May 2004, Volume 14, pp 901-907, Trends in Biotechnology, December 2003, Volume 21, No. 12, pp 531-535, Gen. Res., May 2003, Volume 13, Issue 5, pp 954-964, and Proc. Nat. Acad. of Sci., 2002, Volume 99 (8), pp 5261-5266.

Amplification with phi-29 is linear and may be primed using random primers. The yield of the reaction is limited by the dNTP concentration and not the template concentration because of the very high processivity of the enzyme. The same concentration of product should result regardless of the amount of starting target or PCR amplified target. The primers may have a random region and a constant region.

Bst DNA polymerase is another processive polymerase that is known to have strand displacing activity. The enzyme is available from, for example, New England Biolabs. Bst is active at high temperatures and the reaction-may be incubated, for example at about 65° C. In some embodiments Bst DNA polymerase may be used for templates having increased GC content. The enzyme tolerates reaction conditions of 70° C. and below and can be heat inactivated by incubation at 80° C. for 10 minutes. For additional information see Mead, D. A. et al. (1991) BioTechniques, p.p. 76-87, McClary, J. et al. (1991) J. DNA Sequencing and Mapping, p.p. 173-180 and Hugh, G. and Griffin, M. (1994) PCR Technology, p.p. 228-229.

Any processive DNA polymerases with strand displacing activity may be used. Examples of other enzymes that may be used include: exo minus Vent (NEB), exo minus Deep Vent (NEB), Bst (BioRad), exo minus Pfu (Stratagene), Pfx (Invitrogen), 920 Nₘ™ (NEB), Bca (Panvera), and other thermostable polymerases. Other characteristics of strand displacing enzymes that may be taken into consideration are described, for example, in U.S. Pat. No. 6,692,918.

In many embodiments a method of amplification employing a strand displacing enzyme with high processivity is used to amplify the target. In a preferred embodiment the target has already been enriched in the sample by amplification with PCR using target specific primers. Methods such as multiple displacement amplification (MDA) may be used to amplify the target. MDA and methods of using MDA have been described, for example, in U.S. Pat. Nos. 6,642,034, and 6,617,137.

The target may first be amplified by a template dependent amplification process. In a preferred embodiment PCR is used. PCR is described in detail in U.S. Pat. Nos. 4,683,195, 4,683,202 and 4,800,159, and in Innis et al., 1990. Briefly, two synthetic oligonucleotide primers, which are complementary to two regions of the template DNA (one for each strand) to be amplified, are added to the template DNA, in the presence of excess dNTPs and a thermostable polymerase, such as, for example, Taq DNA polymerase. In a series of temperature cycles, the target DNA is repeatedly denatured (at, for example, around 90° C.) annealed to the primers (at, for example, around 50-60° C.) and a cDNA strand is extended from the primers (at, for example, about 72° C.). As the cDNA strands are created they act as templates in subsequent cycles. Thus, the template region between the two primers is amplified exponentially, rather than linearly.

The yield of a given PCR reaction is influenced by many factors, including the reaction buffer, the magnesium concentration, the sequence and length of the primers and the polymerase used. Also, because the amplification proceeds in an exponential fashion, small differences in template amount in early rounds can result in large differences in amplified product.

In another embodiment nucleic acid samples that are to be pooled for further analysis are normalized by binding each sample to a substrate with the capacity to bind a limiting amount of nucleic acid. The amount of nucleic acid in each sample to be pooled is preferably higher than the capacity of the substrate so an approximately equivalent amount of each sample is bound to the substrate. The substrate bound nucleic acid can then be separated from the substrate and pooled or the substrate and the substrate bound nucleic acid can be pooled.

The substrate may be, for example, a resin, a solid support such as a nylon or paper membrane, or beads. An amount of substrate that has an estimated capacity to bind the desired amount of the amplicon is mixed with the amplicon under conditions that permit binding of the amplicon to the substrate. The excess amplicon may be collected for another use. The bound amplicon may then be subjected to conditions that result in release of the bound amplicon from the substrate and the amplicon can be collected. In a preferred embodiment PCR amplified samples normalized using the methods of the present invention are pooled prior to hybridization to a resequencing array. To obtain optimal performance across the microarray, samples may be pooled to provide an approximately equal number of targets for each probe. The methods may be particularly useful for resequencing analysis using microarrays as described in Cutler et al. Gen. Res. 11:1913-1925, 2001.

In a preferred embodiment templates may be concatenated or circularized to provide longer templates for amplification. For example, after PCR amplification using target specific primers the PCR product may be treated with ligase to allow ligation of two or more amplicons. The PCR may be performed using 5′ phosphorylated primers or the PCR product may be treated with kinase to provide a 5′ phosphate for ligation. Ligation may be by a DNA ligase, for example, T4 DNA ligase or E.coli DNA ligase.

In one embodiment the 5′ ends of the PCR primers include a complementary region so that the ends of the PCR products are complementary and at least one exonuclease resistant base, preferably several, 3′ of the complementary regions. After PCR amplification a 5′ to 3′ exonuclease, for example, T7 gene 6 protein, may be used to digest the 5′ end of the PCR products up to the first exonuclease resistant base. This generates complementary single stranded 3′ overhang on either end of the PCR product. The fragments may then be ligated into concatamers and circles. See, for example, Stoynova et al. (2004) BioTechniques, 36, 402-406.

Strand displacement amplification using a circular template has been described in, for example, Dean et al. Genome Res. 11:1095 (2001). Whole genome amplification using multiple displacement amplification has also been disclosed in Hosono et al. Genome Res. 13, 954-964 (2003) and Yan et al, Biotechniques 37, 136-143 (2004). A single stranded circular nucleic acid may be used as template. One or more primers may be bound to the single stranded circle and initiate synthesis of new strands that are complementary to the circle. The extending strand can displace the primer and previously extended strands from the template. Displaced strands may be used to prime synthesis of new strands.

Genomic restriction fragments may be made into single-stranded circles that can be used as templates. For example, a genomic fragment resulting from digestion with XbaI has the single stranded overhang of CTAG on either end of the fragment. An adaptor can be used to ligate the ends of one strand and introduce one or two gaps into the other strand. The two strands may be denatured to separate because one strand is circular but the other has two free ends. The circular strand may then be used as template by hybridizing a primer, which may be the gapped strand of the adaptor, and extending the primer along the circle. Two or more fragments may be ligated together and joined by a partial adaptor.

In one embodiment genomic DNA is fragmented with one or more restriction enzymes and an adaptor is used to ligate to both ends of fragments to generate a circular molecule comprising the adaptors sequence. The first end of one fragment may be joined to the second end of the same fragment by ligation of both ends to the adaptor, resulting in circularization of the fragment, the first end of one fragment may be ligated to another fragment with an adaptor in between and then the two fragments may be ligated. Two or more fragments may be joined into a long fragment and into a circle in this way. The circles and long fragments may be amplified using rolling circle amplification and strand displacement amplification primed with a primer that is complementary to the adaptor. The adaptor may have a double overhang that is complementary to the ends left by the restriction enzyme or enzymes used. Ligation mediated by the adaptor results in joining of two or more fragments with an adaptor sequence between fragments. The adaptor sequence may be used as a priming site for strand displacement amplification or for rolling circle amplification for circular templates.

In another embodiment a stem loop adaptor sequence may be ligated to each end of a double stranded fragment. The fragment may then be denatured resulting in a single stranded circular fragment that can be used as template for rolling circle amplification. Sequences that are complementary to each strand of the adaptor may be used as primers so that there is a primer that anneals to the original circle and one that anneals to the newly generated copies. In one embodiment, circles that are made only of adaptor sequences without fragment inserts may be digested before amplification by engineering a restriction site that is generated by ligation of two copies of the adaptor. In a preferred embodiment a restriction site such as DrdI is used. The recognition site for DrdI is GACNNNNNNGTC (SEQ ID NO: 1), allowing for the first XbaI site that forms when the adaptor ligates to fragments or to another copy of the adaptor to be present while the DrdI site is generated only when two adaptors are ligated together.

The disclosed methods are particularly well suited to automation. In preferred aspects the targets may be pooled by an automated liquid handling device such as the Capliper Sciclone liquid handling workstation or the Beckman Coulter BIOMEK workstation. Liquid handling devices such as these are particularly well suited to handle large numbers of targets and samples in multi-well plates such as 96 and 384 well plates. Methods for processing multiple microarrays in parallel are disclosed, for example, in U.S. Pat. No. 6,720,149 and in U.S. Provisional application Nos. 60/510,055 and 60/494,891. In preferred aspects automated liquid handling for preparation of labeled target for hybridization to an array may be coupled with automated hybridization to arrays. Arrays may be in a multi-array format analogous to the 96 or 384 well microtitre plate. Such automated systems are commercially available from Affymetrix as the GENECHIP Array Station and facilitate rapid high throughput analysis of a plurality of samples.

Amplification Methods using Circular Templates

The disclosed methods may also be used in conjunction with strand displacement amplification on circular templates as described in Dean et al. Genome Res. 11:1095 (2001). Rolling circle amplification has also been desribed in for example, Fire and Xu, PNAS 92:4641 (1995.) and Liu et al., J. Am. Chem. Soc. 118:1587 (1996)). Sato et al. Biomol Eng. (2005) epub describes use of phi29 and random hexamers for rolling circle amplification. In general the amplification method uses Phi29 polymerase to extend random primers using a circular template. The extended primers are then used as template for subsequent extension of random primers. Because of the strand displacing activity of the polymerase, the reaction can be performed isothermally, without the need to heat denature duplex DNA during amplification. The reaction is limited by time and dNTP concentration not by the concentration of the substrate.

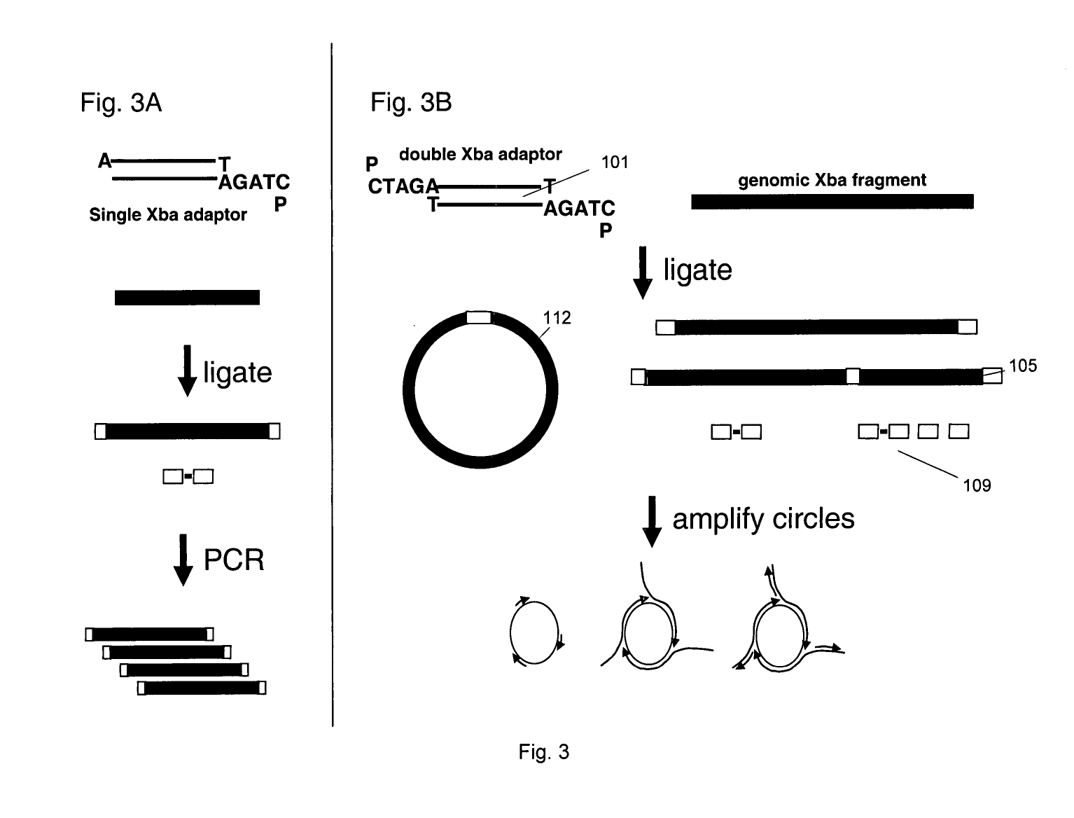

In one embodiment, template, for example genomic fragments, may be circularized by litation of an adaptor with an overhang on either end (FIG. 3). FIG. 3A show a single overhang adaptor. The single Xba adaptor has sticky ends that can ligate to either end of the fragment or to another adaptor to form an adaptor dimer, but does not circularize or form concatamers of fragments or adaptors. The double overhang adaptor [101] (FIG. 3B) can form circularized fragments [112], concatamers of fragments [105] and adaptors [109] and circularized concatamers of fragments. The ligated product can be amplified by a strand displacing polymerase using primers, for example random primers, degenerate primers, or target specific primers. The amplification conditions are preferably limited by the amount of dNTPS or the time so a plurality of reactions can be performed in parallel to give similar amounts of amplified product. See also Wang et al., Genome Res. 14:2357-66 (2004) which uses circularization of fragments followed by RCA as a means of amplifying nucleic acid from samples that are or may be degraded, for example, FFPE samples. In some aspects, the method shown in FIG. 3B is used as the normalizing amplification step because amplification should proceed to completion.

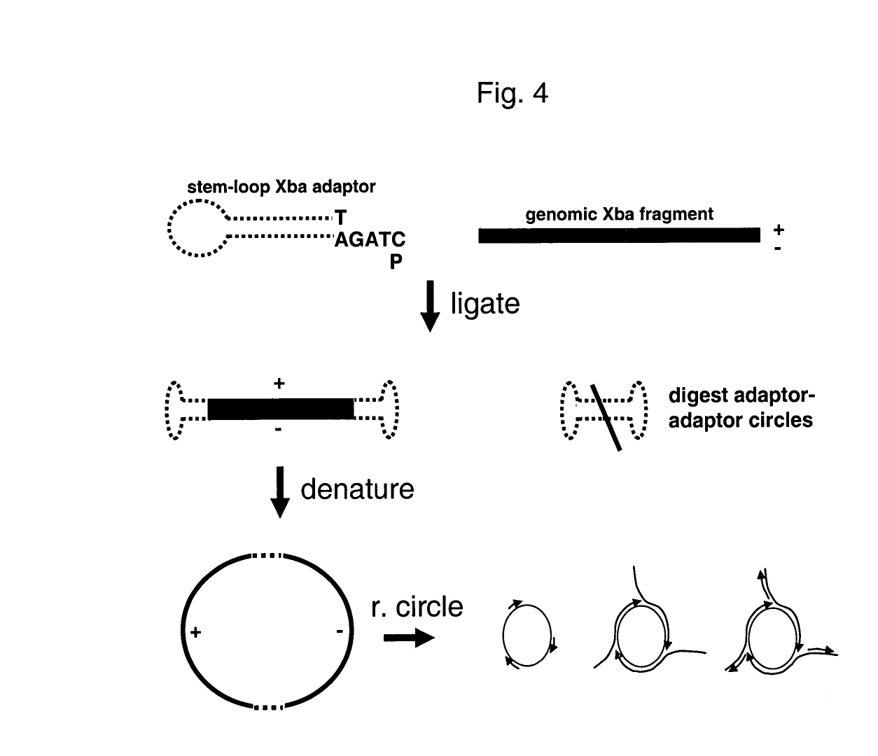

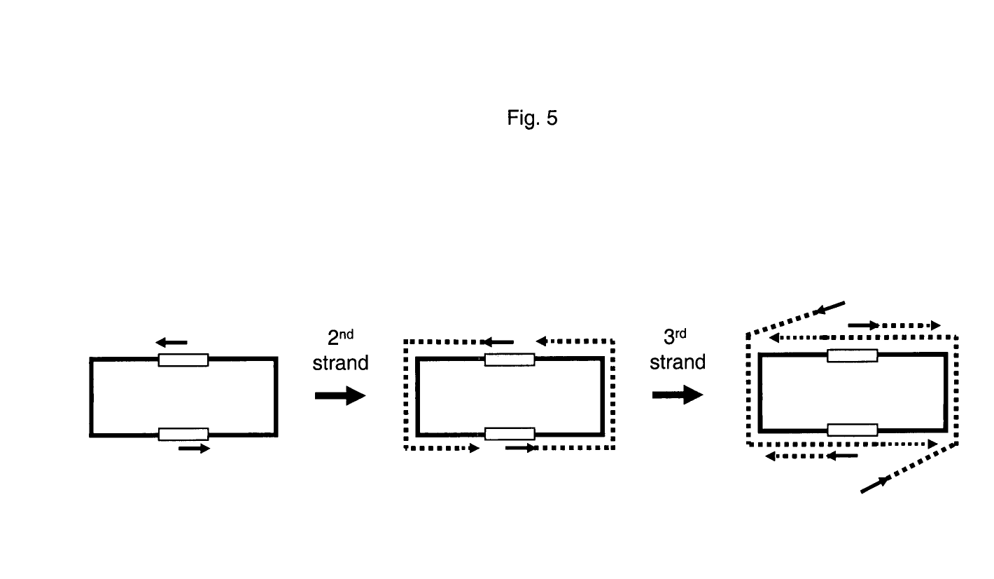

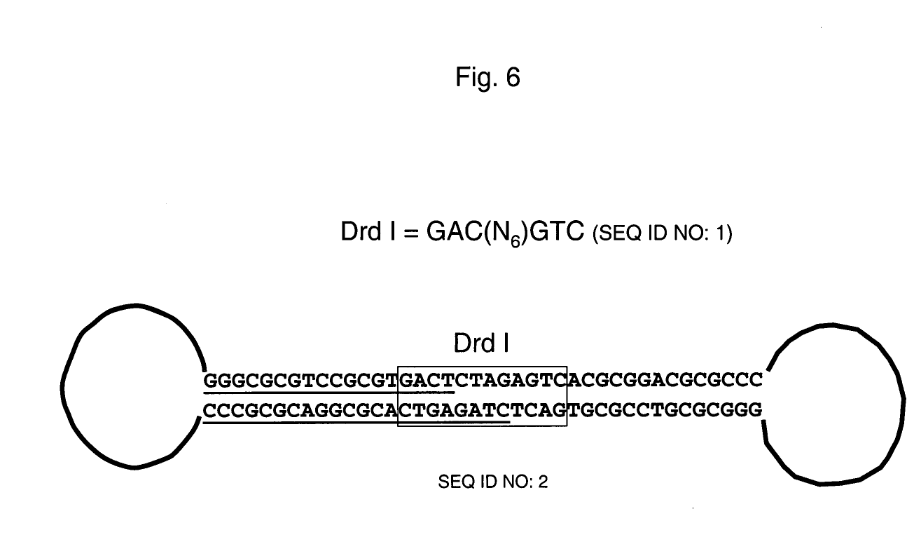

In one embodiment a stem-loop adaptor (FIG. 4) is ligated to genomic fragments to generate a template for RCA. The adaptor can be engineered so that adaptor dimers can be selectively digested without digesting the adaptors that are ligated to genomic fragments. Genomic fragments that have a stem-loop adaptor ligated to both ends can be denatured to generate single stranded circles containing both strands of the genomic fragment. The circles can be used as template in a rolling circle amplification reaction. A primer that is complementary to the adaptor sequence can be used to prime synthesis (FIG. 5). An example of how the adaptors may be designed to introduce a new restriction enzyme when two adaptors ligate together to form a primer dimer is shown in FIG. 6. The restriction site for DrdI is GACNNNNNNGTC (SEQ ID NO: 1). Two identical adaptor sequences ligated together are shown. The sequence of one adapter is 5′CTAGAGTCACGCGGACGCGCCCNₓGGGCGCGTCCGCGTGACT3′ (SEQ ID NO: 2), two copies of the adaptor ligated together are shown. The loop region is Nₓ where X is preferably between 2 and 30 bases.

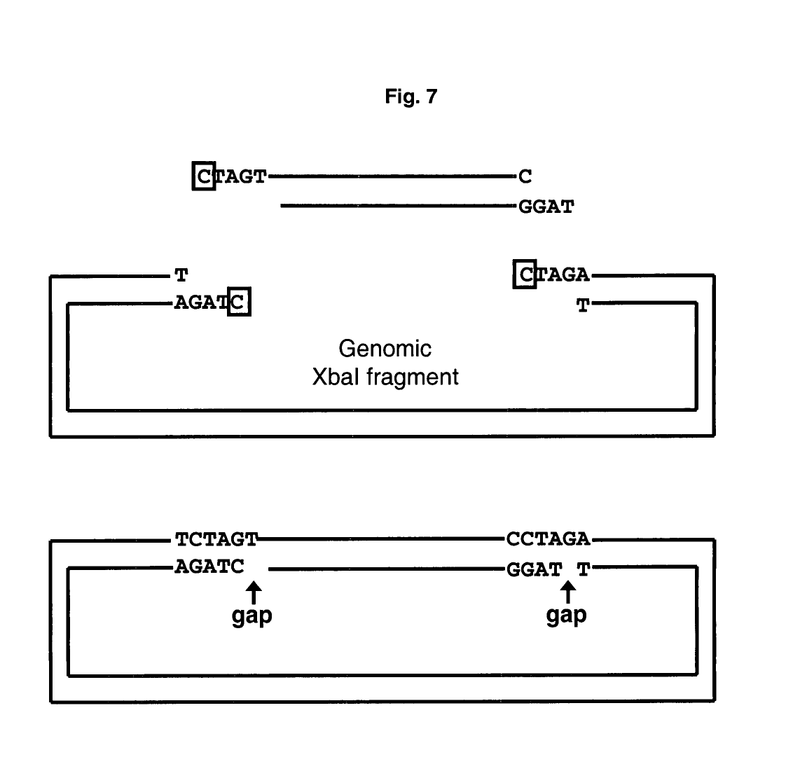

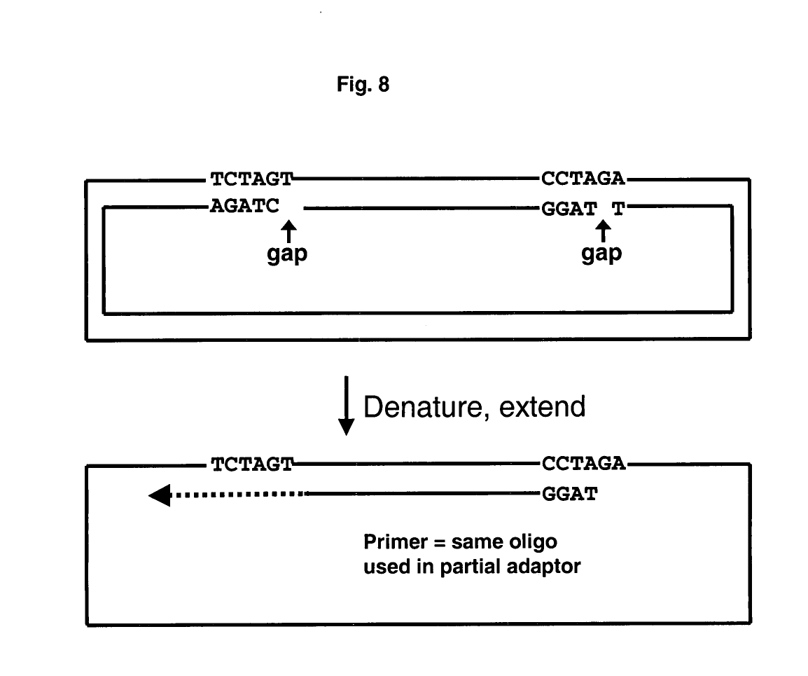

In another embodiment single stranded circular template for amplification by RCA is prepared from genomic fragments by ligating the ends of a fragment together using an adaptor that has a first strand that ligates to both ends of the fragment and a second strand that is not capable of being ligated to the other strand. The second strand may be blocked from ligation by modification or by the introduction of a gap of one or more nucleotides (FIG. 7). The two strands may be denatured and the second strand of the adaptor may be used as a primer to prime synthesis of a copy of the completely circular strand (FIG. 8). Single stranded circles may also be made using an adapter that introduces a gap by blocking ligation by, for example, absence of a phosphate group necessary for ligation. Small filled circles indicate phosphates. Two or more genomic fragments may be joined into a single circle by ligation to the same adaptor. Circles may be formed with two or more genomic fragments and with two or more adaptors.

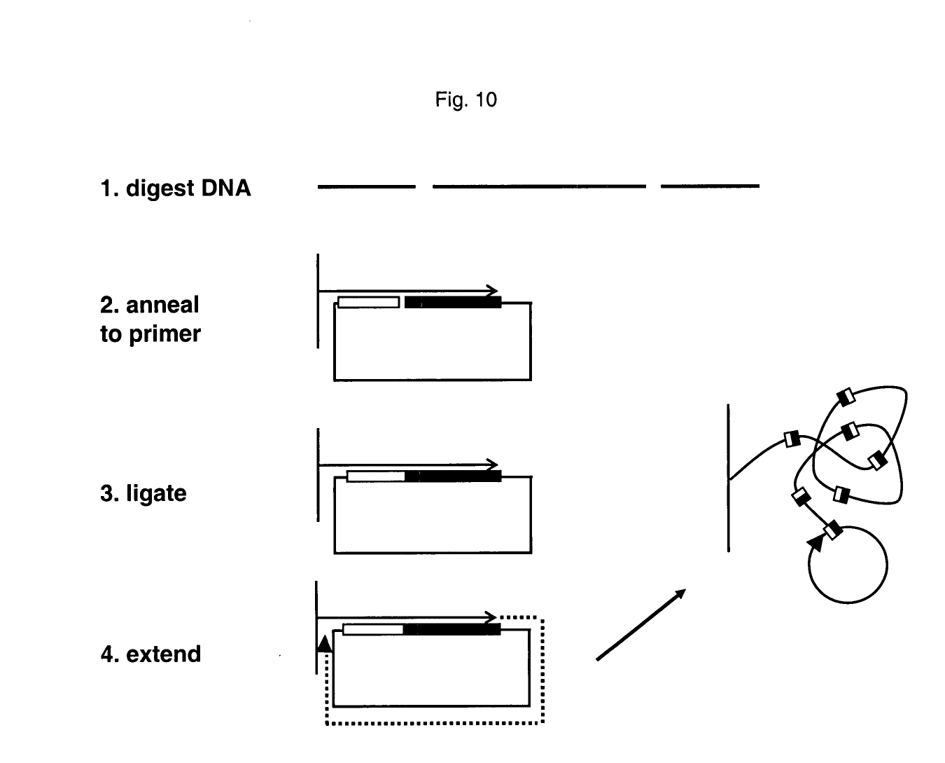

RCA may be used to amplify restriction fragments on a solid support (FIG. 10). Genomic DNA is digested and annealed to a primer attached to a solid support. The ends of a fragment are juxtaposed by the primer on the solid support so that the ends may be ligated. In some embodiments one end is extended so that the ends are juxtaposed for ligation. The primer may then be extended using the circularized fragment as a template in an RCA reaction, generating many copies of the target attached to a solid support.

Genomic DNA may be digested and circularized by ligation of an adaptor that contains a type IIS restriction enzyme. The type IIS enzyme can be used to digest the ligated fragment and it will cut within the genomic fragment. The fragments can be annealed to an array so that the fragments

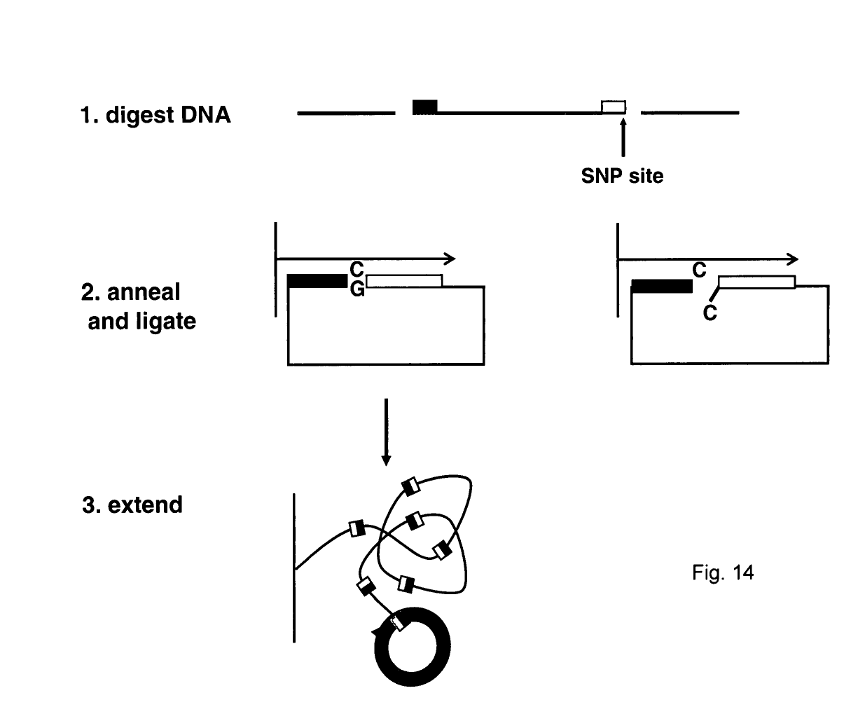

In FIG. 14 a method of genotyping single nucleotide polymorphisms is disclosed. Oligo ligation assay is used to discriminate between alleles. An oligo on the array is complementary to one allele of the SNP and is designed to juxtapose ends of a fragment when the cognate allele is present. If the cognate allele is present the fragment is circularized and RCA can be used to amplify the fragment containing the SNP. The amplified fragment can be detected indicating the presence of the SNP.

### EXAMPLES

### Example 1

Locus specific amplification of long targets. Amplification of genomic DNA may be accomplished in 30 μL PCRs carried out in thin-walled polypropylene tubes or plates using TaKaRa LA Taq (TaKaRa, Biomedicals). The manufacturer's general reaction mixture may be used. Reagents and Materials: LA PCR Kit Ver. 2.1: TakaRa Bio Inc., P/N RR013A; also available from Fisher, P/N TAKRR013A, containing: 10× LA PCR Buffer II (Mg2+): 1 mL/vial, dNTP Mixture: 800 uL/vial, TaKaRa LA Taq: 5 units/μL, Molecular Biology Grade Water: Cambrex, P/N 51200, 1× TE, pH 8: Ambion, P/N 9849 (or other TE); diluted 10-fold in water to give 0.1× TE, 99.9% DMSO: Sigma, P/N D-8418, GeneChip DNA Amplification and Hybridization Control Kit, P/N 900392. Dilute the DMSO to 50% with molecular biology grade water and store at 4° C.

PCR Primers may be purchased from a qualified vendor. Standard salt-free purification is sufficient. Primers should be tested prior to finalizing the array design in order to ensure robust amplification. Re-suspend oligonucleotides in 0.1× TE to create 100 μM stock. The stock can then be stored at −20° C. Create a primer pair stock by combining the 100 μL Forward primer (100 μM), 100 μL Reverse primer (100 μM) and 800 μL 0.1× TE. The final concentration of the diluted stock should be 10 μM for each primer. Aliqout 6 μl of the primer pairs into wells of a 96 well plate.

The genomic DNA used in this assay is preferably of high quality. Particular attention should be paid to ensure that the DNA is free from any PCR inhibitors or proteins. The concentration of the Genomic DNA should be measured by absorbance spectroscopy or by using a reagent such as Picogreen®. Dilute the DNA to 5 ng/μL in molecular biology grade water and store at −20° C.

Add 14 μL of molecular biology grade water to wells of a 96-well plate containing the PCR primers. Each well should now contain: 6 μL primer pair stock and 14 μL molecular biology grade water. Move plate to the PCR Staging Room. Add 20 μL of genomic DNA to each well primer pair mix and water. The total volume of each well should now be 40 μL. Prepare the PCR master mix and keep it on ice to prevent primer degradation from the proofreading activity of the polymerase. Mix is a follows: 33.0 μL water, 16 μL 2.5 mM dNTPs (from TaKaRa Kit) [400 μM final], 10 μL 10× LA PCR buffer(Mg2+) (from TaKaRa Kit), (final 1× buffer and 2.5 μM Mg2+) and 1 μL LA Taq enzyme (from TaKaRa Kit) final concentration is 5 U/100 μL. Total volume is 60 μl.

DMSO is useful in some problematic PCRs. In others, it is unnecessary and even inhibitory. For templates with high GC content, DMSO may be used to a final concentration of up to 5.0% and the volume of water in the reaction reduced accordingly.

Add 60 μL of the PCR master mix to each well. To avoid primer degradation by proofreading enzyme, keep the PCR master mix and DNA-primer plate cold until the thermal cycling reaction starts. Seal the plate. For each reaction: Final Primer concentration=600 nM (each primer), Final DNA template=100 ng/100 /μL

Preheat the PCR block to 94° C. To minimize degradation of the primers by the polymerase, thermal cycling should begin as soon as possible after adding the PCR mix to the DNA/primers. Place the PCR reaction plates in the pre-heated thermal cycler and run the following program: 94° C. for 2 minutes 1×; 94° C. for 10-15 seconds, 68° C. for 1 minute per kb fragment size 30×; 8° C. for 5 minutes+1 minute per kb fragment size 1×, 4° C. HOLD. Verify individual PCR reactions by running 4/μL of each reaction on a 1% TBE agarose gel.

For amplifying autosomal regions, 100 ng of genomic DNA may be used, whereas for X-linked regions, 150 ng may be used. Fragments to be amplified are preferably about 5 to 15 kb long and the yield of a PCR reaction is typically about 10-50 ng/μL.

Following PCR amplification the amplicons may be subjected to a second round of amplification using the REPLI-g Kit from Qiagen. Amplification may be performed according to the instructions in the REPLI-g Handbook (January 2005). Briefly, transfer 2.5 μl of each of the PCR reaction from above is transferred to a new tube (separate tubes for separate reactions). The concentration of the DNA should be at least 4 ng/μl and preferably higher so the 10-50 ng/μl PCR reactions should be sufficient. Add 2.5 μl Buffer D1 to each sample and vortex and centrifuge briefly. Incubate at room temp for 3 min. Add 5 μl Buffer N1 to each sample and mix by vortexing and centrifuge briefly. Thaw REPLI-g DNA polymerase on ice. Thaw all other components at room temp, votex and centrifuge briefly. Prepare a master mix of 27 μl nuclease free water, 12.5 μl REPLI-g buffer, 4×, and 0.5 μl REPLI-g DNA polymerase (volume/reaction). Add 40 μl master mix to 10 μl denatured DNA and incubate at 30° C. for 6 to 16 hours. Inactivate REPLI-g DNA polymerase by heating the sample for 3 min at 65° C. Store DNA at 4° C. or −20° C. for longer storage. For each amplicon calculate the molecular weight and assuming that all tubes have approximately the same number of μg DNA per μl take equal molar amounts of each amplicon and combine in a pooled sample containing about 0.04-0.06 pmoles of each amplicon, preferably about 0.055 moles) in a 35 μl total volume. After pooling the volume may be adjusted to 35 μl with Qiagen EB buffer. The volumes to take from each reaction will depend on the molecular weight of the amplicon, for example, if one amplicon is 2 kb and a second is 4 kb you would need 2 μl of the second to have the same number of moles as 1 μl of the first, assuming that the μg/μl concentration is the same in both. According to the manufacturer of the REPLI-g kit a 50 μl REPLI-g reaction typically yields approximately 40 μg of DNA regardless of the amount of template DNA (see REPLI-g handbook page 18). Fragment, label and hybridize to the array according to the manufacturers instructions.

### Example 2

Rolling Circle amplification. 25 ng XbaI digested genomic DNA was mixed with adaptor, ligase, ATP, NEBuffer 4, DrdI and primers (either 50 or 250 pmol primers) in a reaction volume of either 30 μI or 100 μl. Incubation was at 16° C., then 37° C., then 95° C., then 4° C. Then phil29 polymerase and dNTPs were added and the reaction was incubated at 30° C. for 8 hours. A similar reaction was performed using a stem-loop adaptor. The reaction was incubated for 4, 8 or 16 hours and it was observed that the reaction was complete by 8 hours.

### Conclusion

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herein by reference in their entireties for all purposes.

We claim:

1. A method of amplifying a target sequence from a complex nucleic acid sample comprising:
obtaining a first amplification product that is enriched for the target sequence by incubating the complex nucleic acid sample in a first amplification reaction, wherein said first amplification reaction comprises polymerase chain reaction with a pair of primers that are specific for said target; and,
obtaining a second amplification product by amplifying an aliquot of the first amplification product with a strand displacing DNA polymerase, in the presence of dNTPs and at least one primer in a second amplification reaction.

2. The method of claim 1 wherein the strand displacing DNA polymerase is a phi29 polymerase.

3. The method of claim 1 wherein the yield of the second amplification reaction is limited by the amount of dNTPs added to said second amplification reaction.

4. The method of claim 1 wherein the at least one primer is a collection of random primers.

5. The method of claim 1 wherein the yield of amplified target sequence in the second amplification reaction is about 1 to 2 μg of amplified target sequence per μl reactionn volume.

6. The method of claim 1 wherein the yield of amplified target sequence in the second amplification reaction is about 0.5 to 1 μg of amplified target sequence per μl reaction volume.

7. The method of claim 1 wherein the strand displacing polymerase is Bst DNA polymerase.

8. A method of obtaining a pooled sample comprising approximately equal molar amounts of a plurality of amplified target sequences comprising:
(a) amplifying each target sequence according to the method of claim 1 wherein the amount of dNTPs present in the second amplification of each target sequence is approximately the same;
(b) obtaining an estimate of the molecular weight of each target;
(c) determining a volume of the second amplification reaction to add to a pooled sample for each of the targets, so that each will be present at approximately the same molar amount in the pooled sample, using the estimated molecular weight of each target and assuming that the amount of DNA in each of the second amplification reactions is the same; and
(c) obtaining the pooled sample by mixing the volumes of the second amplification reaction calculated in (c) to a new tube.

9. The method of claim 8 wherein the yield of amplified target sequence in the second amplification reaction for each target sequence in the plurality of amplified target sequences is about 0.5 to 1 μg of amplified target sequence per μl reaction volume.

10. The method of claim 8 wherein the yield of amplified target sequence in the second amplification reaction for each target sequence in the plurality of amplified target sequences is about 1 to 2 μg of amplified target sequence per μl reaction volume.

11. The method of claim 8 wherein the volume of the second amplification reaction added to the pooled sample is proportionate to the molecular weight of the target in said second amplification reaction.

12. The method of claim 8 wherein each target is between 1 and 30 kilobases in length.

13. The method of claim 8 further comprising analyzing the pooled sample by fragmenting the targets in the pooled sample to generate fragments, labeling the fragments to generate labeled fragments and hybridizing the labeled fragments to a resequencing array.

14. The method of claim 8 wherein an automated liquid handling device is used for mixing the volumes of the second reaction.

15. A method for obtaining a pooled sample comprising approximately equimolar amounts of a first amplified target sequence and a second amplified target sequence comprising
Amplifying said first target sequence in a first amplification reaction to generate a first amplification product wherein the first target is amplified by PCR with a pair of primers that are specific for said first target sequence;
amplifying said second target sequence in a second reaction to generate a second amplification product wherein the second target sequence is amplified by PCR with a pair of primers that are specific for said second target sequence;
amplifying an aliquot of said first amplification product in a third amplification reaction, to generate a third amplification product, wherein the third amplification reaction comprises a mixture of random primers, a strand displacing DNA polymerase, and a first amount of dNTPs;
amplifying an aliquot of said second amplification product in a fourth amplification reaction, to generate a fourth amplification product, wherein the fourth amplification reaction comprises a mixture of random primers, a strand displacing DNA polymerase, and a second amount of dNTPs; and
mixing a volume of the third amplification product with a volume of the fourth amplification product to generate a mixture of amplified first and second target sequences wherein the first and second target amplicons are present in approximately equal molar amounts in the mixture.

16. The method of claim 15 wherein said strand displacing DNA polymerase is selected from the group consisting of a phi29 polymerase and a Bst polymerase.

17. The method of claim 15 wherein said first amount of dNTPs and said second amount of dNTPs are approximately equal.

18. The method of claim 15 wherein said first amount of dNTPs is proportional to the molecular weight of the first target sequence and said second amount of dNTPs is proportionate to the molecular weight of the second target sequence.

19. The method of claim 18 wherein the volume of the third amplification product and the volume of the fourth amplification product that are added to the mixture are approximately equal.

20. The method of claim 15 wherein the first target sequence and the second target sequence are between 1 and 5 kilobases in length.

21. The method of claim 15 wherein the first target sequence and the second target sequence are between 5 and 15 kilobases in length.

22. The method of claim 15 wherein the yield of the third amplification product and the yield of the fourth amplification product are approximately equal.


SEQUENCE LISTING (2 sequences)

SEQ ID NO: 1 - DNA, 12 bp, Artificial
  Endonuclease DrdI restriction sequence
gacnnnnnngtc

SEQ ID NO: 2 - DNA, 42 bp, Artificial
  Endonuclease DrdI restriction sequence engineered within a stem loop structure
ctagagtcacgcggacgcgcccnngggcgcgtccgcgtgact